(19) United States
(12) Patent Application Publication
(10) Pub. No.: US 2018/0140468 A1
(43) Pub. Date: May 24, 2018

(54) Glove and Sock Burn Dressing with Variable Pore Sizes

(71) Applicant: E. Aubrey Woodroof, Carlsbad, CA (US)

(72) Inventor: E. Aubrey Woodroof, Carlsbad, CA (US)

(21) Appl. No.: 15/396,720

(22) Filed: Jan. 2, 2017

Related U.S. Application Data
(60) Provisional application No. 62/424,436, filed on Nov. 19, 2016.

(51) Int. Cl. A61F 13/10; A61M 35/00; A61L 15/26; A61L 15/44; A61L 15/42

(52) U.S. Cl. CPC A61F 13/104; A61M 35/00; A61M 2207/00; A61L 15/44; A61L 15/425; A61L 15/26

(57) ABSTRACT

An improved glove-shaped dressing for hands is presented which enables quicker wound closure and a lower anesthesia requirement, comprised of two hand-shaped pieces of an improved prior-art skin-substitute brought together with a sealing means and fitted to the hand with surgical staples.

### RELATED APPLICATIONS

This application claims the benefit of U.S. Provisional Patent Application 62/424,436, which is included here by reference.

### FIELD OF THE INVENTION

This invention relates to dressings and bandages for wounds, specifically for hand burns.

### BACKGROUND OF THE INVENTION

Wound management involves removal of all non-viable tissue at the wound site, preserving the remaining viable tissue, and providing a moist but not wet environment. An example of successful burn wound dressing is Biobrane, granted U.S. Pat. No. 4,725,279. In 1979 Biobrane was initially studied by American Burn Surgeons; it is still popular world-wide.

In 2007 new art was introduced by the present inventor with AWBAT and then with AWBAT Plus, granted U.S. Pat. No. 7,815,931 and covered by several copending patent applications. The key to the success of the AWBAT or AWBAT Plus Glove products was: better porosity in the dressing and a three part glove for right or left hand enabling better anatomical fit—necessary for hospital to inventory right and left handed gloves.

The present invention uses a new material called Permeaderm® that allows passage of fluid from the wound through the primary dressing (this invention) into a secondary absorbent dressing (separate and not this invention) as well as improving the kinetics of uninterrupted wound healing. Technology of this dressing has evolved into a new product which possesses all the characteristics and attributes known to be important for optimal wound healing, as well as containing certain advances that result in minimization of wound desiccation and infection complication. Key advantages of this technology is multifold: very rapid closure of the hand burn by the clinician while providing excellent fit and conformance to the highly irregular wound surface of the hand, and at a lower cost. A single PermeaDerm® Glove works for either the right or left hand which reduces the cost of hospital inventory.

### SUMMARY OF THE INVENTION

The present invention is a new approach to providing a glove bandage that addresses burn injuries. The present invention enables faster and better wound closure to fitting thumb and 3 fingers initially; then the clinician completes closure using greater circumferential elongation of the PermeaDerm® material plus extra material on distal portion of hand and ‘pinkie’ finger. Result is an almost perfect anatomical fit for the hand reducing operating room time and general anesthesia.

Burn hand wound sites have variable amounts of: exudate, transudate, and plasma present. The clinician must cleanly debride the wound, close it and manage wound healing in a moist but not wet environment to achieve optimal results. This is particularly difficult with complicated structures like hands and feet. A hydrophilic/hygroscopic moist, but not wet, wound healing environment is known to enhance healing. The Permeaderm® material is a more sophisticated biological coating than competing materials such as Biobrane or AWBAT/AWBAT Plus.

The present invention provides a dressing that possesses all the properties and attributes of an ideal skin substitute and, in addition, has ‘variable porosity’ controlled by the clinician from zero porosity to what the wound requires. The present invention enables the clinician to move the fluid exuding from the wound through the primary dressing into an absorbent secondary dressing without disturbing the kinetics of healing or causing pain to the patient.

The present invention is composed of a single layer of biologicals over the 3D nylon/silicone surface with an average 21.4 micrograms of gelatin per sq. cm. with a standard deviation of 2.4 and an average 52.8 micrograms of Aloe per sq. cm with a standard deviation of 6.3.

In vitro, the Aloe component has been demonstrated to cause a variety of cells to attach and proliferate; as well as increase synthesis of collagen and alpha smooth muscle actin. ECM may be added to the biologicals described above and is a mixture from human fibroblasts that is known to cause rapid cell proliferation and tissue growth. Previous wound dressings and skin substitutes, as taught in U.S. Pat. No. 7,815,931 contain gelatin, a pure Aloe component, chondroitin 4 & 6 sulfate, and vitamin C & E. In contrast the current dressing will have one layer of biologicals with about 20 micrograms of gelatin per sq. cm. and about 50 micrograms of Aloe per sq. cm.

### BRIEF DESCRIPTION OF THE FIGURES

FIG. 1 is a display of a typical glove pattern stamped out of a sheet of Permeaderm.

FIG. 2 is a diagram showing how two mirror pieces of the glove are mated

FIG. 3 shows an assembled glove with sealed edges

FIG. 4 shows a completed glove with a human hand inserted, with staples around the closing edge of the glove

FIG. 5 shows a piece of Permeaderm® with the mirrored glove patterns imposed on it

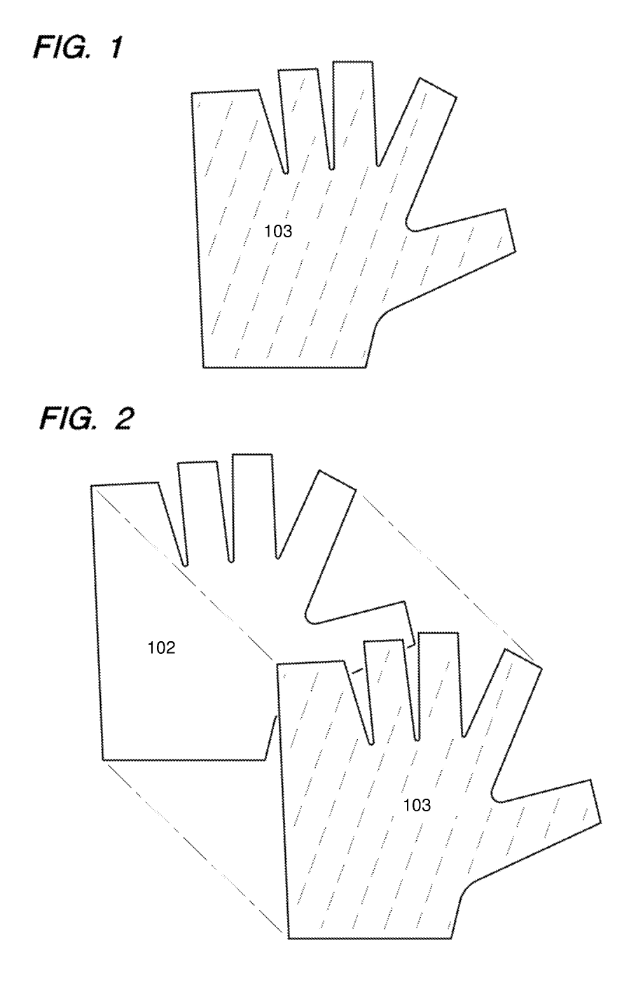

### DETAILED DESCRIPTION

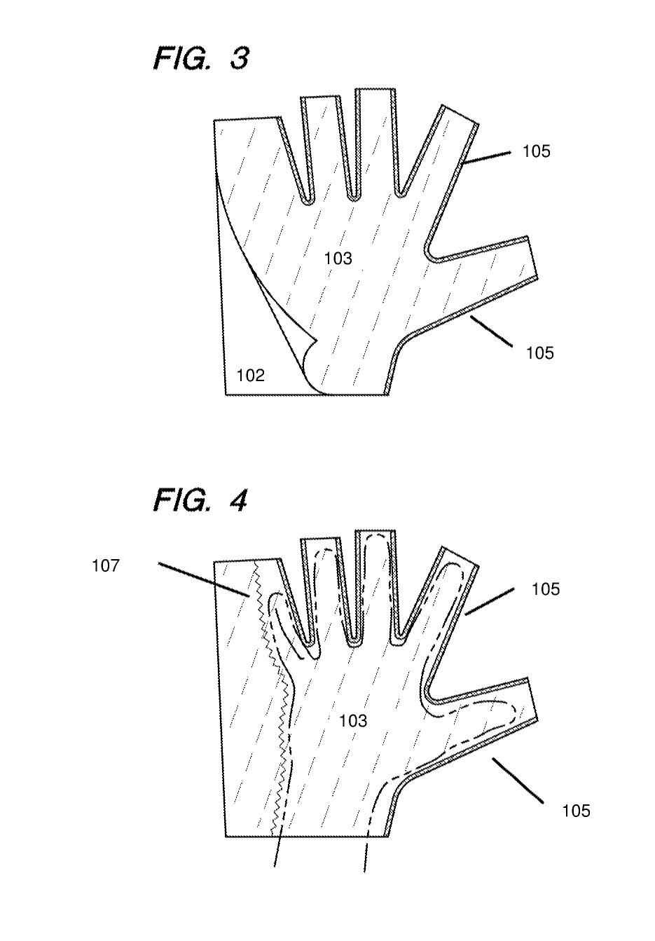

The present invention is a glove made by glueing two pieces of PermeaDerm® together with the biologically coated 3D nylon structure 102 facing each other and the slitted silicone surface 103 facing out, as shown in FIG. 2; the 3D coated nylon surface 102 contacts the wound surface inside the glove. For lower cost and efficiency, glue is preferred to seams sewn together with thread. As seen in FIG. 3, the little finger is left open and unsealed initially when the glove is fitted to the hand, and is closed later with surgical staples 107.

The present invention is similar in composition to earlier skin substitutes in that they each have a thin silicone component and an underlying thin knitted nylon component. The present invention differs from its ancestors in that it has “variable porosity” controlled by the clinician; the slit 104 width in the thin silicone will be essentially zero (with no stretch, in relaxed mode) to a higher porosity (proportional to the stretch applied). In the Figures, the slit 104 widths are emphasized for visibility.

In addition, the present invention differs from prior art in the composition of biological coatings applied to both components and how these coatings interact with the wound over time.

The pores of prior art skin substitutes/dressings are of a fixed size (Biobrane 1.2%; AWBAT and AWBAT Plus 5.5% and 7.5%) in the unstretched open position; the silicone is cured while the skin substitute pores are open. Once cured the pores cannot close or be reduced in size; this causes wound desiccation and punctate scarring. In the present invention using Permeaderm®, in contrast, the openings are made after the silicone component has been cured, and are in the shape of slits, not holes.

The preferred embodiment of the invention designed for burns, a regular pattern with slits 104 on the silicone surface 103 parallel to each other in the same row is presented. The slits made in the silicone are approximately 0.125″ long with a space of 0.50″, between the slits; off-set parallel rows of slits are 0.25″ apart.

In this configuration the silicone/nylon membrane can be stretched in a direction perpendicular to the slit orientation and the slits 104 will open. Porosity therefore increases proportionally to the amount of stretch applied. Obviously, there is a maximum amount of stretching of the preferred embodiment before the dressing fails.

The use of the present invention has a large benefit because it is stable on the wound and possesses biologicals that aid in the healing process.

The present invention will have a single layer of biologicals comprised of a hypoallergenic BSE free USP Pharmaceutical grade gelatin, pure Aloe or Aloesin, pure Aloe and BSE free gelatin, or a mixture of pure Aloe, BSE free gelatin and ECM interact with the wound to stimulate the rate of healing while adherent to the wound. The layer is deposited directly on the nylon side 102 of the “variable porosity” silicone/nylon surface and is stable, i.e. requires 100 degree water for 30 minutes to remove from the “variable porosity” silicone/nylon surface.

The knitted nylon component 102 would be the thinnest commercially-available mesh, the preferred material nylon. Either 12/1 or 15/1 denier filament would be used to knit the mesh.

The biologicals are applied to the knitted nylon side 102 of the present embodiment. Optionally, the biologicals can be applied to the silicone layer 103 as well.

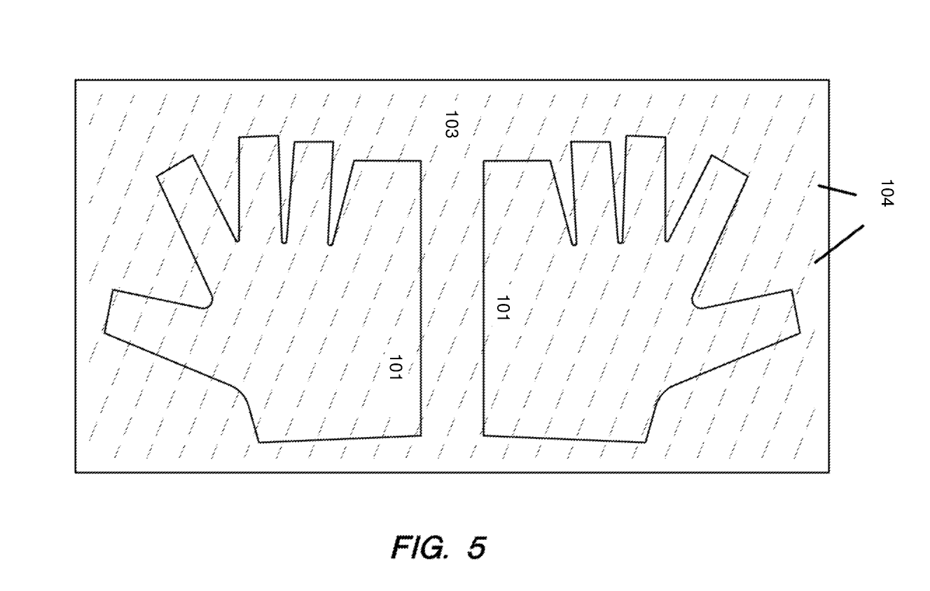

A preferred method of manufacture for the present invention is to stamp out a plurality of glove sizes, presently estimated at five different hand sizes from pediatric up to full adult, from large sheets of pre-manufactured Permeaderm® B material. One size pattern is shown in FIG. 5. These hand blanks 101 are then sealed at their edges as in FIG. 3 and FIG. 4, with the exception of the little finger, with a sealing means 105.

As shown in FIG. 4. the little finger edge of the glove will be closed 107 with medical staples along the side of the hand. Additionally, the ends of the fingers of the glove will be closed with medical staples (not shown).

The preferred sealing means is restricted medical grade silicone room-temperature vulcanization (RTV), with a preferred bead width of ⅛ inch. The medical grade glue does not have to be sterile, because the assembled invention(s) are passed through intense electron beam radiation as a final manufacturing step, which sterilizes the entire structure.

This is the preferred embodiments of the invention. The technology to create the invention is listed in the preferred embodiments of this invention, but other methods are possible and are within the contemplation of this patent.

What is claimed is:

1. A glove-shaped hand dressing, the hand dressing comprised of two hand-shaped pieces of a skin substitute,
The two hand-shaped pieces cut out from a single sheet of said skin substitute, the hand-shaped pieces mirror images of each other and the patterns of the hand-shaped pieces placed side-by-side on said single sheet,
the skin substitute comprised of two layers of material,
the first layer of material comprised of a silicone membrane, the second layer comprised of a woven fabric,
the two layers combined together with a combination means such that the two layers form a single structure, the preferred combination method is heating,
the first layer possessing a plurality of slits in its surface, said slits made after the two layers are combined, said slits in a regular pattern, the regular pattern comprising a parallel vertical orientation,
said first layer and said second layer treated with a one or more medicinal or therapeutic substances,
the porosity of said skin substitute minimized in the mode where no stretching tension is exerted on the skin substitute, the porosity of said skin substitute variable proportional to the amount of stretching tension and the direction in which said stretching tension is placed on the skin substitute.

2. The skin substitute of claim 1 where the plurality of slits are each approximately 0.125 inch long and arranged in a plurality of rows.

3. The skin substitute of claim 1 where the medicinal and therapeutic substances are selected from the list of hypoallergenic BSE free USP Pharmaceutical grade gelatin, pure aloe, aloesin and ECM.

4. A method of assembling a glove-shaped hand dressing as in claim 1, the method comprised of the steps of
laying a two-glove, mirrored pattern on a single sheet of skin substitute,
cutting out each glove pattern into a glove blank,
placing the two glove blanks on top of each other such that the second layers comprised of woven fabric are facing each other,
sealing the thumb edge of the glove and the finger edges with a sealing means, excepting the little finger of the glove.

5. A method of using a glove-shaped hand dressing as in claim 1 and claim 4, comprised of the steps of
obtaining an assembled glove as generated in claim 4,
carefully sliding a human hand into said assembled glove leaving the little finger edge of the glove open,
stapling the ends of the fingers of said assembled glove with surgical staples,
then at an appropriate time later, stapling the little finger edge of the assembled glove together with surgical staples.
